(19) United States
(12) Patent Application Publication
(10) Pub. No.: US 2010/0050793 A1
(43) Pub. Date: Mar. 4, 2010

(54) FLEXIBLE CHEMICAL SENSORS

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/200,802

(22) Filed: Aug. 28, 2008

(51) Int. Cl. G01N 33/00

(52) U.S. Cl. 73/866

(57) ABSTRACT

A flexible sensor device can include a flexible substrate, and at least one flexible sensor included on the flexible substrate. The flexible sensor can be deposited on the flexible substrate by inkjet printing a composition that forms the flexible sensor. The flexible sensor device can be configured to function when subjected to elongation, contraction, and/or distortion.

### BACKGROUND

The detection of one or more specific chemical entities in a composition, as well as in a particular environment, has been a goal in various technical areas. Medical diagnostics have employed the detection of specific entities, such as insulin, cancerous cell receptors, and the like, in order to determine the presence or progression of a disease state. Military and homeland security organizations have employed the detection of specific chemical agents in order to screen for agents that may be hazardous (e.g., poisons, explosives, and the like) or may be precursors for hazardous materials (e.g., reagents for making drugs, poisons, explosives, and the like). While a wide range of detection technologies exists, as evidenced by the medical and security devices currently employed, many of the detection equipment and procedures lack in sensitivity and/or efficient usability. Additionally, developments in the ideology of biosensors has led to the search for suitable sensors to detect biological chemicals for various reasons, including the monitoring of a subject's health.

Recently, many different types of sensor strategies have been devised and employed. These sensors can have various configurations and structures and/or materials that have the ability to generate a signal when stimulated by a specific type or genus of stimuli. Often, the sensors are configured to be chemosensors that sense the interaction of a specific type or genus of a substance with a recognition substrate associated with the sensor. Typically, the sensors are configured to sense electronic, optical, magnetic, and/or electrochemical signal changes upon recognition of a target substance. The output of the sensors can be measured for detection of one or more specific substances.

In many cases, the available sensors and sensor technologies are not compatible with many uses because of the physical configuration of the sensors. In part, the adaptability of sensor to different environments has been an obstacle for sensor development.

### SUMMARY

Generally, a flexible sensor device can include a flexible substrate, and at least one flexible sensor included on the flexible substrate. The flexible sensor can be deposited on the flexible substrate by inkjet printing a composition that forms the flexible sensor. The flexible sensor can be configured to function when subjected to elongation, contraction, and/or distortion.

In one embodiment, the flexible sensor can be a macrosensor that includes one or more sensors. Also, the flexible sensor can include one or more nanosensors. The flexible sensor can be configured to detect a target substance so as to provide a detectable signal. Non-limiting examples of target substances can include an organic molecule, inorganic molecule, atom, ion, nucleotide, polynucleotide, amino acid, polypeptide, protein, receptor, antibody, antibody fragment, cell, cell surface component, ligand, or combinations thereof.

In one embodiment, the flexible sensor is a flexible sensor circuit. As such, the flexible sensor circuit can be inkjetted onto the flexible substrate in the pattern of a desired sensor circuit. As such, components that form a flexible circuit can be inkjet printed onto the substrate so as to form the flexible sensor circuit. In one aspect, a flexible sensor circuit includes at least one of a nanowire or a conducting polymer. In one aspect, the flexible sensor circuit includes at least one nanowire and at least one conducting polymer.

In one embodiment, the flexible sensor device can be configured for being included in a garment. Accordingly, the flexible substrate and/or the inkjetted sensor can be configured with sufficient flexibility for being a component of a wearable garment such that the sensor is capable of sensing a target substance that provides biometric data of a subject wearing the wearable garment.

The flexible sensor device can be prepared by a method of manufacturing that utilizes inkjet printing and inkjet printing systems. Such an inkjetting method can include selecting a flexible substrate, and inkjetting at least one flexible sensor onto the flexible substrate. Additionally, the flexible sensor device can be achieved by configuring the flexible substrate having the flexible sensor to function as a sensor device when subjected to elongation, contraction, and/or distortion. Additionally, the flexible sensor can be configured to function as a sensor when subjected to elongation, contraction, and/or distortion. The flexible sensor can be formed by inkjetting a plurality of sensors onto the flexible substrate to form a flexible macrosensor. Also, the flexible sensor can be formed by inkjetting one or more nanosensors onto the flexible substrate. The flexible sensor can be configured to detect a target substance so as to provide a detectable signal.

In one embodiment, the flexible sensor can be formed into a flexible sensor circuit. As such, the method of manufacturing the flexible sensor device can include inkjetting one of a nanowire or a conducting polymer onto the flexible substrate to form the flexible sensor circuit. Alternatively, the method of manufacturing the flexible sensor device can include inkjetting a nanowire and a conducting polymer onto the flexible substrate to form the flexible sensor circuit.

These and other embodiments and features of the sensor device will become more fully apparent from the following description and appended claims, or may be learned by the practice of the sensor device as set forth hereinafter.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic representation of an illustrative embodiment of a flexible sensor device having flexible sensors.

FIG. 2 is a schematic representation of an illustrative embodiment of a flexible sensor device having flexible sensor circuits.

FIG. 3 is a schematic representation of an illustrative embodiment of a flexible sensor device having flexible sensor circuits in electronic communication.

FIG. 4 is a schematic representation of an illustrative embodiment of a flexible sensor device having flexible sensor circuits that are configured for being electronically coupled to an external device.

FIG. 5 is a schematic representation of an illustrative embodiment of a printing system for printing a flexible sensor device having flexible sensors.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Generally, flexible sensor devices and compositions for making and using the same can be used for detecting the presence of a target substance. The flexible sensor devices and compositions can be configured to include various concentrations or amounts of flexible sensors that interact with the target substance to provide a detectable signal as an indication of such an interaction. The flexible sensor device can be achieved by placing one or more sensors or sensor circuits onto a flexible substrate that holds and retains the one or more sensors or sensor circuits. The flexible substrate can have various configurations that provide for sufficient flexibility for an intended use while retaining the functionality of the one or more sensors or sensor circuits. Discussions of sensors are intended also to refer to sensor circuits and vice versa.

The flexible sensor device can include one or more sensors or sensor circuits on a flexible substrate. The amount of one or more sensors or sensor circuits can vary. Accordingly, the flexible sensor device can include one or more sensors and/or sensor circuits. However, the sensor device can include about or at least about 10 sensors and/or sensor circuits, at least about 50 sensors and/or sensor circuits, at least about 100 sensors and/or sensor circuits, at least about 1,000 sensors and/or sensor circuits, at least about 10,000 sensors and/or sensor circuits, at least about 100,000 sensors and/or sensor circuits, at least about 1 million sensors and/or sensor circuits, at least about 10 million sensors and/or sensor circuits, least 100 million sensor and/or sensor circuits, or at least 1 billion sensors and/or sensor circuits. Also, the sensor device can include from about 1 nanosensor about 10 nanosensors, from about 10 nanosensors to about 50 nanosensors, from about 50 nanosensors to about 100 nanosensors, from about 100 nanosensors to about 1,000 nanosensors, from about 1,000 nanosensors to about 10,000 nanosensors, from about 10,000 nanosensors to about 100,000 nanosensors, from about 100,000 nanosensors to about 1 million nanosensors, from about 1 million nanosensors to about 10 million nanosensors, from about 10 million nanosensors to about 100 million nanosensors, or from 100 million nanosensors to about 1 billion nanosensors. The number of sensors and/or sensor circuits included on the flexible substrate may be limited by the surface area available. Thus, the size of the flexible substrate can limit the number of sensors and/or sensor circuits, depending on the surface area density of the sensors and/or sensor circuits as well as the size of the inkjet printed sensors.

A flexible sensor device can be configured to be used for detecting a target substance in a medium. The flexible sensor device can include a flexible substrate, and at least one flexible sensor included and retained on the flexible substrate. The sensor can be configured to interact with a target substance so as to provide a signal that can be detected. The target substance can be any type of substance. Non-limiting examples of a suitable target substance can include an organic molecule, inorganic molecule, atom, ion, nucleotide, polynucleotide, amino acid, polypeptide, protein, receptor, antibody, antibody fragment, cell, cell surface component, ligand, combinations thereof, or the like. When the target substance is a target polynucleotide, the sensor can include a probe polynucleotide configured to hybridize with the target polynucleotide. When the target substance is a target polypeptide, the sensor can include a target recognition moiety configured to interact with the target polypeptide. When the target substance is a target cell, the sensor can include a target recognition moiety configured to interact with a cell surface component of the target cell. Non-limiting examples of cell surface components include a protein, epitope, receptor, cell membrane component, lipid, combinations thereof, or the like.

In one embodiment, a flexible sensor device that detects polynucleotides can include at least one flexible sensor that detects polynucleotides included and retained on a flexible substrate. The flexible sensor can include a probe polynucleotide configured to hybridize with a target polynucleotide. Also, the probe polynucleotide of the nanosensor can have a high degree of specificity for the target polynucleotide, the high degree of specificity being characterized by at least 90% complementarity.

As used herein, the terms “complementary” and “complementarity” are meant to refer to the ability of polynucleotides to form base pairs with one another. Base pairs are typically formed by hydrogen bonds between nucleotide units in anti-parallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine.

Perfect complementarity or 100% complementarity refers to the situation in which each nucleotide unit of one polynucleotide strand can hydrogen bond with a nucleotide unit of an anti-parallel polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. “Substantial complementarity” refers to polynucleotide strands exhibiting 79% or greater complementarity, that are selected so as to be non-complementary.

In one embodiment, a flexible sensor device that detects polypeptides can include at least one flexible sensor that detects polypeptides included and retained on a flexible substrate. The sensor can include a target recognition moiety configured to interact with a target polypeptide. The target recognition moiety can be, but is not limited to, one of a polypeptide, protein, receptor, antibody, antibody fragment, ligand, combinations thereof, or the like. The target recognition moiety can be selected and/or configured to interact with the target poloypeptide in any possible condition or manner.

In one embodiment, a flexible sensor device that detects cells can include at least one flexible sensor that detects cells included and retained on a flexible substrate. The sensor can include a target recognition moiety configured to interact with a cell surface component of a target cell. Non-limiting examples of a cell surface component include a protein, epitope, receptor, cell membrane component, lipid, combinations thereof, or the like. The target recognition moiety can be selected and/or configured to interact with the target cell in any possible condition or manner.

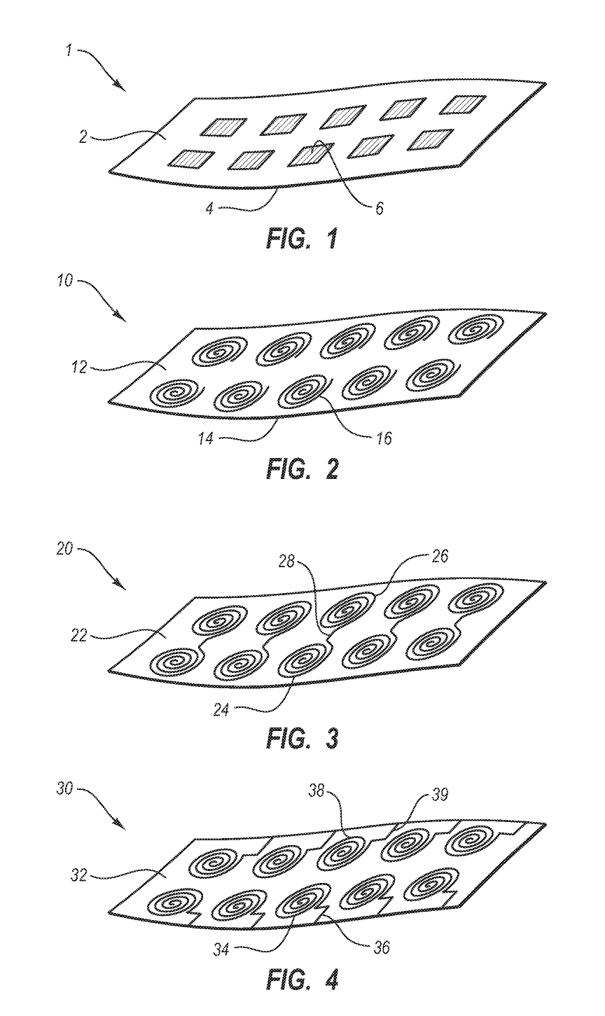

FIG. 1 illustrates an embodiment of a flexible sensor device 1. The flexible sensor device 1 can have a flexible substrate 2 with a surface 4 that is configured for receiving a flexible sensor 6. The flexible sensor 6 can be any flexible sensor or sensor circuit that can detect the presence of a target substance. The substrate 2 can be made of a polymeric body and/or an inorganic-organic complex. Also, ceramics with suitable flexibility can be included in the substrate. Examples of suitable materials for inclusion in the substrate are described below.

The flexible substrate 2 can have any suitable shape or dimension along any vector. The flexible substrate 2 can also be a porous substrate. The pores (not shown) can extend, for example, from the surface 4 into the substrate 2 or all the way through the substrate 2. The shape shown for the substrate 2 is substantially flat-rectangular; however, other shapes are possible. Non-limiting examples of the shape of the substrate 2 can include a block, triangle, amorphous shape, sphere, cube, polygon, and the like formed in three dimensions or as a substantially two dimensional sheet.

The pores (not shown) can be configured to form at least one conduit that opens to the outside of the surface 4 of the substrate 2 or to the sensor 6 and extends to a location within the substrate 2 or all the way through the substrate 2. The pores can be any type of pores or pore system, or other similar configuration that allows for a substance to pass therethrough. The pores can be shaped, sized, and/or dimensioned to perform size exclusion selection on the substances that can pass therethrough. That is, the pores can be configured to restrict substances of a certain size from entering into the pores and/or passing from one surface 4 of the substrate 2 to the opposite surface. Accordingly, the pores allow substances smaller than a certain size to enter into the pores. The size of the pores can be configured to be similar to the target substance, which can restrict access to the nanosensors and increase the accuracy of detection when the substrate is used for size exclusion selection. Non-limiting examples of pores sizes include being about, or less than about 0.1 nm, less than about 1 nm, less than about 10 nm, less than about 100 nm, less than about 1 um, less than about 10 um, and less than about 100 um. Additional non-limiting examples of pores sizes include being about 0.01 nm to about 0.1 nm, about 0.1 nm to about 1 nm, about 1 nm to about 10 nm, about 10 nm to about 100 nm, about 100 nm to about 1 um, about 1 um to about 10 um, and about 19 um to about 100 um.

FIG. 2 illustrates an embodiment of a flexible sensor device 10 with sensor circuits. The flexible sensor device 10 can have a flexible substrate 12 with a surface 14 that is configured for receiving a flexible sensor circuit 16. The flexible sensor circuit 16 can be any flexible sensor circuit that can detect the presence of a target substance. The substrate 12 can be made of a flexible polymeric body and/or an inorganic-organic complex. The substrate 12 is shown to include more than one sensor circuit 16 that are individual sensors. As such, the substrate 12 can be partitioned so that the smaller substrate only includes one sensor circuit 16. The sensor circuit 16 can be a combination of sensors, nanowires, conductive polymers, and the like, and can include target recognition moieties for detecting target substances.

FIG. 3 illustrates an embodiment of a flexible sensor device 20 with a complex sensor circuit. The flexible sensor device 20 can have a flexible substrate 22 that is configured for receiving a first flexible sensor circuit 24 that is electronically coupled to a second flexible sensor circuit 26. Such electronic coupling can be obtained, for example, an electronic path 28 operatively linking the first flexible sensor circuit 24 and the second flexible sensor circuit 26. The electronic coupling of flexible sensor circuits 24, 26 can be used to prepare more complex sensor systems. Also, any number of sensor circuits can be electronically coupled. The sensor circuits can be configured as described herein.

FIG. 4 illustrates an embodiment of a flexible sensor device 30 with sensor circuits that can be coupled to an external device, such as a monitoring device or computing system. The flexible sensor device 30 can have a flexible substrate 32 that is configured for receiving a first flexible sensor circuit 34 that is electronically couplable to an external device through a first electronic path 36. Additionally, the flexible substrate can include a second flexible sensor circuit 38 that can be electronically coupled to the same or other external device through a second electronic path 39. The first flexible sensor circuit 34 and second flexible sensor circuit 38 can be configured to detect the same or different chemical substances. The electronic paths 36, 39, can allow for the flexible sensor circuits 34, 38 to be capable of providing data to the external device. The electronic coupling of flexible sensor circuits 34, 38 with an external device can be used to prepare more complex sensor systems, such as those that can monitor or detect different chemical substances. Also, any number of sensor circuits can be electronically coupled. The sensor circuits can be configured as described herein.

The flexible substrate can be prepared from any polymer. This can include non-biocompatible polymers as well as biocompatible polymers. In one instance, the biocompatible polymer can be a biostable polymer. In another instance, the biocompatible polymer can have a degree of biodegradability. Non-limiting examples of general polymers that can be configured for suitable flexibility for use in a flexible sensor device can include: polyethylenes, polyethylene (PE), Low density polyethylene (LDPE), high density polyethylene (HDPE), crosslinked polyethylene (XLPE); polypropylenes, polypropylene (PP), polybutylene (PB), polyisobutylene (PIB), biaxially-oriented polypropylene; polyarylates, polymethyl methacrylate (PMMA), polymethyl acrylate (PMA), hydroxyethyl methacrylate (HEMA), polybutadiene acrylonitrile (PBAN), sodium polyacrylate polyacrylamide (PAM); polyesteres, polystyrene (PS), polyethylene terphthalate (PET), acrylonitrile butadiene styrene (ABS), high impact polystyrene (HIPS), extruded polystyrene (XPS); polysulphones, polysulfone (PSU), polyarylsulfone (PAS), polyethersulfone (PES), polyphenylsulfone (PPS); polyamides (PA), polyphthalamide (PPA), bismaleimide (BMI), urea formaldehyde (UF); polyurethanes (PU), polyisocyanurate (PIR); polyvinyls, polyvinyl chloride (PVC), polyvinylidene chloride (PVDC); fluoropolymers, fluoroethylene (FE), polytetrafluoroethylene (PTFE); ethylene chlorotrifluoroethlyene (ECTFE); polycarbonate (PC), polylactic acid (PLA), and the like. Non-limiting examples of biocompatible polymers that can be used in the flexible sensor device can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, copolymers thereof, derivative polymers thereof, monomers thereof, combinations thereof, or the like. Other biocompatible, biodegradable, and/or biostable polymers can be used with or in place of any of the above-referenced polymers. The flexible substrate can also be water stable so that the container body does not degrade in the presence of water or other aqueous solution. Also, the flexible substrate can be prepared from polymers that have stability in organic solutions so that the flexible sensor device does not degrade when in an organic solution, organic components, or hydrophobic components.

Non-limiting examples of inorganic-organic complexes that can be included in flexible substrates can include: flexible ligand 1,3-bis(4-pyridyl)propane with Co(NCS)₂.xH₂O; a combination of a sulfonate salt and an alkaline inorganic metal salt, whereby the crystalline structure of the inorganic portion of the complex is platelet and film-forming in character; organic-inorganic coordination polymers, [Cd(3-pmpmd)(CH₃CN)₂(H₂O)₂]ₙ.2n(ClO₄)₂ and [Zn(3-pmpmd)_{1.5}(H₂O)₂]ₙ.2n(ClO₄)₂.nCH₃CN, can be obtained from M(ClO₄)₂ (M=Cd, Zn) and the semi-flexible 3,3′-N-donor bis-pyridyl ligand 3-pmpmd (N,N′-bis(3-pyridylmethyl)pyromellitic diimide); polyethylene terephthalate (PET) inorganic complexes, SiOₓ/PET and AlOₓ/PET substrates; [Zn(Meen)₂]₂[(4,4′-bipy)Zn₂As₈V₁₂O₄₀(H₂O)], [Zn(en)₂(H₂O)][Zn(en)₂(4,4′-bipy)Zn₂As₈V₁₂O₄₀(H₂O)].3H₂O, [{Zn(en)₃}₂{Zn₂As₈V₁₂O₄₀(H₂O)}].4H₂O.0.25bipy, and [Zn₂(en)₅]{[Zn(en)₂][(bpe)HZn₂As₈V₁₂O₄₀(H₂O)]₂}.7H₂O [en=ethylenediamine, Meen=1,2-diaminopropane, 4,4′-bipy=4,4′-bipyridine, and bpe=1,2-bis(4-pyridyl)ethane] can be constructed from organically modified Zn-substituted polyoxovanadates and zinc organoamine subunits; or a titania/isostearate nanocomposite self-standing film with high transparency and flexibility prepared via a sol-gel process, in which a titanium tetraisopropoxide/isostearate complex (precursor), n-hexylammonium isostearate (catalyst), and o-xylene (solvent) were used. The sol obtained by the sol-gel reaction was floated on a water surface to form an unsupported film.

The flexible sensor 6, as shown in FIG. 1, can be any sensor or combination of sensors as well as sensor circuits. The flexible sensor 6 can be a single sensor or a combination of sensors, such as combination of nanosensors. The sensor 6 can be configured to detect a chemical substance, such as but not limited to, organic molecule, inorganic molecule, atom, ion, nucleotide, polynucleotide, amino acid, polypeptide, protein, receptor, antibody, antibody fragment, cell, cell surface component, ligand, combinations thereof, or the like.

In one embodiment, the sensor or sensor circuit can be configured to detect a target polynucleotide. Such a sensor can include a probe polynucleotide that is configured for hybridizing or otherwise associating with a target polynucleotide. The interaction between the probe polynucleotide and the target polynucleotide can provide a signal that can be detected. The probe polynucleotide can have a high degree of specificity for the target polynucleotide, the high degree of specificity being characterized by at least about 75%, at least about 90%, or at least about 99% complementarity of the target polynucleotide with the probe polynucleotide, or about 50% to about 75%, about 75% to about 90%, or 90% to about 99% complementarity. The interaction between the target polynucleotide and probe polynucleotide can provide a signal that is selected from the group consisting of an electronic signal, optical signal, magnetic signal, electrochemical signal, and combinations thereof. Also, the interaction between the target polynucleotide and probe polynucleotide of the nanosensor can induce a detectable change in the signal.

In one embodiment, the sensor or sensor circuit can be configured to detect a target polypeptide. Such a sensor can include a target recognition moiety configured for binding, associating, or interacting with a target polypeptide. The target recognition moiety can be, for example without limitation, a protein, receptor, antibody, antibody fragment, or the like that interacts with a target polypeptide. The sensor can have a high degree of specificity for the target polypeptide, wherein high specificity can be characterized by the target recognition moiety only interacting with the target polypeptide, medium specificity can be characterized by the target recognition moiety interacting with the target polypeptide and derivatives and analogs thereof, and low specificity can be characterized by the target recognition moiety interacting with a genus of polypeptides that include the target polypeptide as a species thereof. Also, the interaction between the sensor can provide a signal selected from the group consisting of an electronic signal, optical signal, magnetic signal, electrochemical signal, and combinations thereof. Also, the interaction between the target recognition moiety and the target polypeptide can induce a detectable change in the signal.

In one embodiment, the sensor or sensor circuit can be configured to detect a target cell. Such a sensor can include a target cell recognition moiety (e.g., protein, receptor, antibody, antibody fragment, ligand, etc.) that interacts with a cell surface component of the target cell. Non-limiting examples of a cell surface component include a protein, epitope, receptor, cell membrane component, lipid, combinations thereof, or the like. The sensor can have a high degree of specificity for the target cell, wherein high specificity can be characterized by the target recognition moiety only interacting with the target cell, medium specificity can be characterized by the target recognition moiety interacting with the target cell and other similar cell types, and low specificity can be characterized by the target recognition moiety interacting with a genus of cells that include the target cell as a species thereof. Also, the interaction between the sensor can provide a signal selected from the group consisting of an electronic signal, optical signal, magnetic signal, electrochemical signal, and combinations thereof. Also, the interaction between the target recognition moiety and the target polypeptide can induce a detectable change in the signal.

The sensors and/or sensor circuits that can be included in the flexible sensor devices described herein represent a broad class of sensors that can be employed to detect a target substance. The sensors can include those described herein as well as those well known in the art and those later developed.

In one embodiment, a sensor or sensor circuit can include a nanowire. Such nanowires have high surface-to-volume ratios, and can be synthesized from ceramics and polymers. The nanowires can be used to detect chemical agents (e.g., pesticides), microorganisms (e.g., E. coli, Giardia), and mineral compounds (Nanobiotechnology: The promise and reality of new approaches to molecular recognition; Fortina et al.; Trends In biotechnology; Vol. 23, No. 4, April 2005). The nanowires can include surface or other interfacial chemical modifications to achieve selectivity for a target substance. As such, receptors, ligands, epitopes, antibodies, antibody fragments, and the like can be included on nanowires.

A nanowire is a wire of a diameter of the order of a nanometer, and can be defined as structures that have a lateral size constrained to tens of nanometers or less and an unconstrained longitudinal size. Many different types of nanowires exist, including metallic nanowires (e.g., Ni, Pt, Au, etc.), semiconducting nanowires (e.g., Si, InP, GaN, etc.), and insulating nanowires (e.g., SiO₂,TiO₂, etc.). Molecular nanowires can include repeating molecular units including either organic (e.g. DNA, RNA, etc.) or inorganic (e.g. Mo₆S₉₋ₓIₓ) components. Nanowires can have aspect ratios of about 1000 or more. As such, nanowires can be referred to as 1-Dimensional materials. Electrons in nanowires are quantum confined laterally, and thus occupy energy levels that are different from the traditional continuum of energy levels or bands found in bulk materials. Quantum confinement of certain nanowires, such as carbon nanotubes, can provide electrical conductance. Non-limiting examples of nanowires can include inorganic molecular nanowires (e.g., Mo₆S₉₋ₓIₓ, Li₂Mo₆Se₆), which have a diameter of 0.9 nm, and can be hundreds of micrometers long. Additional non-limiting examples of nanowires can be based on semiconductors (e.g., InP, Si, GaN, etc.), dielectrics (e.g. SiO₂,TiO₂), or metals (e.g. Ni, Pt).

Nanowires can be used to fabricate sensor circuits by chemically doping a semiconductor nanowire to create p-type and n-type semiconductors. Also, a p-n junction, one of the simplest electronic devices, can be prepared by physically crossing a p-type wire over an n-type wire or chemically doping a single wire with different dopants along the length. Additionally, nanowires can be fabricated into logic gates by connecting several p-n junctions together, which provide a basis for all logic circuits: the AND, OR, and NOT gates can be prepared from semiconductor nanowire crossings.

In one embodiment, a sensor circuit can include a conducting polymer. Conducting polymers are configured to allow electrons to flow across so as to be electrically conductive. The conducting polymers can be used to prepare sensor circuits similarly to the use of conducting materials in circuits. Non-limiting examples of conducting polymers that can be used to prepare sensor circuits can include: conductive polypyrrole; high conductivity oxidized iodine-doped polypyrrole, a polyacetylene derivative; poly(phenylene vinylene) (PPV), which is an alternating copolymer of polyacteylene and poly(paraphenylene) can be a semiconducting polymer; poly(3-alkylthiophenes); a self-doped mixed copolymer of oxidized polyacetylene, polypyrrole and polyaniline having near metallic conductivity; organic conductive polymers, poly(acetylene), poly(pyrrole), poly(thiophene), poly(aniline), poly(fluorene), poly(3-alkylthiophene), polytetrathiafulvalene, polynaphthalene, poly(p-phenylene sulfide), poly(para-phenylene vinylene); malanins; derivatives thereof; combinations thereof; or other conducting polymers.

In one embodiment, a sensor or sensor circuit includes a molecule or ion sensor. Such molecular sensors can be configured to detect the presence of specific substances, and combine the properties of supramolecular receptors, as they specifically recognize a specific substance, with the ability to produce a measurable signal. Optical signals based on changes of absorbance, transmission, or fluorescence are the most frequently utilized because of their simple applications and use of common instruments. The molecular sensors can change absorbance, particularly of color, when interacting with a target substance. Such changes can be used to detect the presence of the target substance. The use of molecular sensors that provide or change fluorescence emission provides very high sensitivity of the sensor device. One category of fluorescence chemosensors includes classical fluorescence chemosensors made from molecules in which a supramolecular receptor and a fluorescence dye are part of the same molecule. Another class is that of self-organized fluorescence chemosensors, which are obtained by the spontaneous self-organizing of the sensor components.

A fluorescence chemosensor, ATMCA, can be obtained by coupling an anthrylmethyl group to an amino nitrogen of TMCA (2,4,6-triamino-1,3,5-trimethoxycyclohexane), a tripodal ligand selective for divalent first-row transition metal ions in water. The ATMCA ligand can act as a versatile sensor for Zn and Cu ions, where the sensing ability can be switched by simply tuning the operating conditions. At pH 5, ATMCA detects copper ions in aqueous solutions by the complexation-induced quenching of the anthracene emission. Metal ion concentrations <1 μM can be readily detected and very little interference is exerted by other metal ions. At pH 7, ATMCA signals the presence of Zn ions at concentrations <1 μM by a complexation-induced enhancement of the fluorescence. Such a chemosensor is a nanosensor, and can be used in the sensor devices as described herein.

Additionally, the [Zn(ATMCA)]₂₊ complex can act as a fluorescence nanosensor for specific organic species, such as selected dicarboxylic acids and nucleotides, by the formation of ternary ligand/zinc/substrate complexes. The oxalate anion can be detected in concentrations <0.1 mM. Nucleotides containing an imide or amide function can be detected with the nanosensor, and the nanosensor has high sensitivity for guanine derivatives. Moreover, the ATMCA.Zn(II) complex is an effective and selective sensor for vitamin B13 (orotic acid) in sub-micromolar concentrations. The formation of the complex with vitamin B13 leads to the quenching of the fluorescence emission of anthracenyl residue.

Another non-limiting example of a nanosensor is a Foster resonance energy transfer (FRET) amplified chemosensor. The sensing activity includes the binding of Al(III) to a 3,5-bis(ortho-hydroxyphenyl)-1,2,4-triazole group, and produces a chelation induced fluorescence enhancement (CHEF). The 3,5-bis(ortho-hydroxyphenyl)-1,2,4-triazole group can be used as a sensor as described herein. Also, conjugation of the 3,5-bis(ortho-hydroxyphenyl)-1,2,4-triazole group with coumarine 343 allows the amplification of the fluorescence signal via a FRET process.

Another non-limiting example of a nanosensor is a self-assembled chemosensor for Cu(II) having decylglycylglycine and ANS chromophore in close proximity. The Cu(II) selective receptor (decylglycylglycine) and a chromophore (ANS) can be in close proximity with CTABr surfactant so as to aggregate. Also, the components can be coupled to a microparticle, such as silica. The close proximity produces fluorescence quenching after Cu(II) addition in concentrations below the micromolar range. Commercially available particles (e.g., 20 nm diameter) can be functionalized with triethoxysilane derivatives of selective Cu(II) ligands and fluorophores. The sensor components can be coupled to the particle surface to provide spatial proximity to signal Cu(II) by quenching of the fluorescence emission. In 9:1 DMSO/water solution, the coated silica nanoparticles (CSNs) selectively detect copper ions down to nanomolar concentrations, and the operative range of the nanosensor can be tuned by the simple modification of the components ratio.

A tren-based tripodal chemosensor bearing a rhodamine and two tosyl groups can be prepared as a sensor to detect metal ions. Detection can be observed through UV/vis and fluorescence spectroscopies. Addition of a Hg²⁺ ion to the nanosensor can provide a visual color change as well as significantly enhanced fluorescence, while other ions including Pb²⁺, Zn²⁺, Cu²⁺, Ca²⁺, Ba²⁺, Cd²⁺, Co²⁺, Mg²⁺, Ag⁺, Cs⁺, Li⁺, and Na⁺ induced no or much smaller color/spectral changes. As such, the sensor is an Hg²⁺-selective fluorescent sensor. Such a nanosensor can be used as described herein.

Additionally, quantum dots or barcode quantum materials having specific arrangements and fluorescent augmentations can be used in a nanosensor. Zinc sulfide quantum dots, though not quite as fluorescent as cadmium selenide quantum dots, can have augmented fluorescence by including other metals such as manganese and various lanthanide elements. The quantum dots can become more fluorescent when they bond to their target, such as target substances, polynucleotides, polypeptides, and cells. The quantum dots or barcode quantum materials having the quantum dots can be used in ultrasensitive nanosensors. Different high-quality quantum dot nanocrystals (ZnS, CdS, and PbS) can be tagged to a target recognition moiety (e.g., probe polynucleotides, ligands, receptors, antibodies, antibody fragments, etc.) for on-site voltammetric stripping measurements of multiple antigen targets. The quantum dots or barcode quantum materials can have distinct redox potential and yield highly sensitive and selective stripping peaks at −1.11 V (Zn), −0.67 V (Cd) and −0.52 V (Pb) at a mercury-coated glassy carbon electrode compared to references. The change in position and size of these peaks reflect the presence and concentration level of the corresponding target.

A nanosensor can include a nanotube having a target recognition moiety that interacts with a target substance, polynucleotide, polypeptide, or cell. Accordingly, the target recognition moiety is configured for interacting with the target. The nanotube, such as a carbon nanotube, can have a first vibrational energy when the target recognition moiety is not interacting with the target and then have a second vibrational energy when the target recognition moiety interacts with the target. The difference between the first and second vibrational energy is measurable and detection of the difference can provide an indication that the target is present. Thus, any type of target recognition moiety can be applied to a nanotube in order to have a sensor that can be used as described herein. Energies other than vibrational energy may also be used for detection purposed.

In one embodiment, a nanosensor can be configured as a “core-satellite” structure, which resembles a planet (gold) with numerous smaller moons (particles) tethered to it by tiny strands of polynucleotides having probe polynucleotide sequences. The probe polynucleotide sequences can be configured for hybridizing with the target polynucleotide so as to have suitable complementarity. Gold core particles and smaller satellite particles of various materials are mixed together in solution with the probe polynucleotides and under controlled circumstances assemble themselves into the desired core-satellite structure. Following assembly, the structures are can be used to detect new strands of polynucleotides of various lengths. The probe polynucleotide tethers between the gold core and particles contract or expand when in the presence of the target polynucleotide. As the particles move in relation to the gold core, the optical properties of the structure change, and thereby provide a signal that can be detected.

In one embodiment, a nanosensor can be a bio-barcode nanosensor. A bio-barcode nanosensor includes a nanosensor that includes a series of barcode oligonucleotides. The barcode oligonucleotides can correspond to a specific target, and interaction of the target with the nanosensors releases one or more of the bio-barcodes, which can be detected.

In one embodiment, a nanosensor can include a nano-gap capacitor. Nan-gap capacitors can be fabricated using silicon nanolithography. A target recognition moiety is immobilized on the nano-gap capacitor in a manner that allows for interaction with the target substance. When the target substance interacts with the target recognition moiety, the capacitance changes in a detectable manner. As such, the nano-gap capacitor is configured to change the detected signal upon interaction of the target substance and a nanosensor.

In one embodiment, a nanosensor can include a nano-cantilever. A target recognition moiety is immobilized on the nano-cantilever in a manner that allows for interaction with the target substance. When the target substance interacts with the target recognition moiety, the deflection properties, vibrational properties, or response to probe signals changes in a detectable manner. Thus, a nano-cantilever can be coupled to a target substance recognition moiety such that interaction of the target substance and the recognition moiety changes the detected signal of the nano-cantilever.

In one embodiment, a sensor system can include any sensor device as described herein that includes a nanosensor in a polymeric container as described herein, and can include a monitor configured to detect a signal that indicates the nanosensor has sensed the target substance. The monitor can be selected based on the type of signal provided by the nanosensor.

The flexible sensors or sensor circuits on the flexible substrate can be configured to have various shapes and sizes over a broad range. With regard to size, the flexible sensors or sensor circuits can have a dimension, such as diameter, width, length, height, or the like, that ranges from about 10 nm to about 1 mm. In another option, the dimension can range from about 50 nm to about 100 um. In yet another option, the dimension can range from about 75 nm to about 10 um. In still yet another option, the dimension can range from about 100 nm to about 1 um. Also, larger flexible substrates can range between the foregoing values in the micrometer (um) range, millimeter (mm) range, and centimeter (cm range), or larger if needed. In some instances certain applications can utilize flexible sensors or sensor circuits that are larger, equal to, or smaller than any of the recited dimensions.

The flexible sensors or sensor circuits can have a high degree of specificity for the target substance. This can include the flexible sensors or sensor circuits being specific for the target substance so that the signal is provided only when the flexible sensors or sensor circuits interacts with the target substance, which is an example of strict specificity. Also, less stringent specificity can be used where the flexible sensors or sensor circuits provides the signal when it interacts with the target substance or a close derivative, analog, salt, or other minor change. Loose specificity can be used when the flexible sensors or sensor circuits provides a signal when interacting with one of a member of a class or a species of a genus of types of target substances.

Flexible sensors or sensor circuits can be configured to provide a signal that is selected from the group consisting of an electronic signal, optical signal, magnetic signal, electrochemical signal, and combinations thereof. Accordingly, a flexible sensors or sensor circuits can be selected or manufactured based on the type of signal provided. In different instances, any of the above-references signal types can be favorable. The selection of the flexible sensors or sensor circuits may result in a specific type of signal in instances where the flexible sensors or sensor circuits interact with a target substance to provide a specific signal type.

The flexible sensors or sensor circuits can provide a signal having a first characteristic in the absence of the target substance and then change the signal to a second characteristic upon interaction with the target substance. This can include a first wavelength or first wavelength pattern that is changed to a second wavelength or second wavelength pattern. The signal can have an absorption, transmission, or other emission profile that has a first characteristic, and the characteristic is changed to a second characteristic upon interaction with the target substance. Such a change can be detectible so that the detection of the targets substance results from detection in a change in the signal from a first characteristic to a second characteristic.

The flexible sensor device having the flexible sensors and/or sensor circuits can be configured for any degree of flexibility. This can include having sufficient flexibility to be bent from being flat to 180 degrees so as to be folded over itself. Also, the flexible sensor device can be rolled into a sleeve, tube, or the like. Additionally, the flexible sensor device can be configured to have sufficient flexibility to be included in a garment in any location of the garment, such as locations at the knee, buttocks, waste, abdomen, armpits, shoulders, elbows, and the like. Accordingly, the flexible sensor device and/or the flexible sensors and/or flexible sensor circuits can have any degree of elongation, contraction, and/or distortion. For example, without limitation, the flexibility can allow for elongation and/or distortion so as to change a dimension, such as length, width, height, diameter, or the like by about 110%, about 135%, about 150%, about 175%, about 200%, about 500%, or to about 1000% of the original value of the dimension, wherein 100% would be considered no change. In another non-limiting example, the contraction and/or distortion can change a dimension by about 90%, about 80%, about 75%, about 60%, about 50%, about 30%, about 25%, about 15%, or about 10% of the original value.

In one embodiment, a method of detecting a target substance with a flexible sensor device can be performed with a flexible sensor device as described herein that includes a flexible sensor or sensor circuit. The flexible sensor device can be placed in a medium to determine whether or not the target substance is present. When the sensor or sensor circuit of the flexible sensor device interacts with a target substance, a signal is provided. As such, detecting the signal provides an indication that the presence of the target substance in the medium. Optionally, the medium can be selected from the group consisting of water, air, biological sample, hydrocarbon, skin, tissue, body fluids, combinations thereof, and other similar media.

Additionally, the method can further include tagging the target substance with a marker that interacts with the sensor device so as to provide the signal. In various systems, a donor and acceptor can be used as a marker pair, where the target substance is modified to include one of the donor and acceptor and the sensor has the other. Close proximity or association of the donor and acceptor provides the detectable signal. For example, a target nucleic acid can be tagged with the marker, which is either the donor or acceptor, and the probe polynucleotide has the other. When the target hybridizes with the probe, the signal is provided.

The method of detecting a target substance can also include determining an amount or concentration of the target substance in the medium. Quantification of the signal or change in signal can be used to determine the amount or concentration of the target substance. Also, the signal can be compared to a control or control set in order to quantify or quantitate the amount or concentration of the target substance.

The method of detecting a target substance can include the use of a probe signal that induces the detection signal to be provided or to change the signal. As such, a probe signal can be directed into the medium to the nanosensor so as to induce at least one nanosensor to provide the signal. The probe signal can provide energy that is changed by the nanosensor in a detectable manner. For example, light of a broad or specific wavelength can be directed into the medium, and the obtained absorbance, transmittance, or fluorescence can be the signal provided as a result of the probe signal.

### VII. Manufacturing Sensor Devices

The sensor devices as described herein can be prepared by various methods of depositing, printing, or otherwise including a flexible sensor or flexible sensor circuit on a flexible substrate. The substrate can include a flexible polymer or inorganic-organic complex, which substrate can be porous in some instance. In other instances, the substrate can be substantially devoid of pores.

In one embodiment, a method of manufacturing a flexible sensor device can be performed by inkjetting. The inkjetting method can use an inkjet printer or other similar printing device or system that can print a composition onto a substrate.

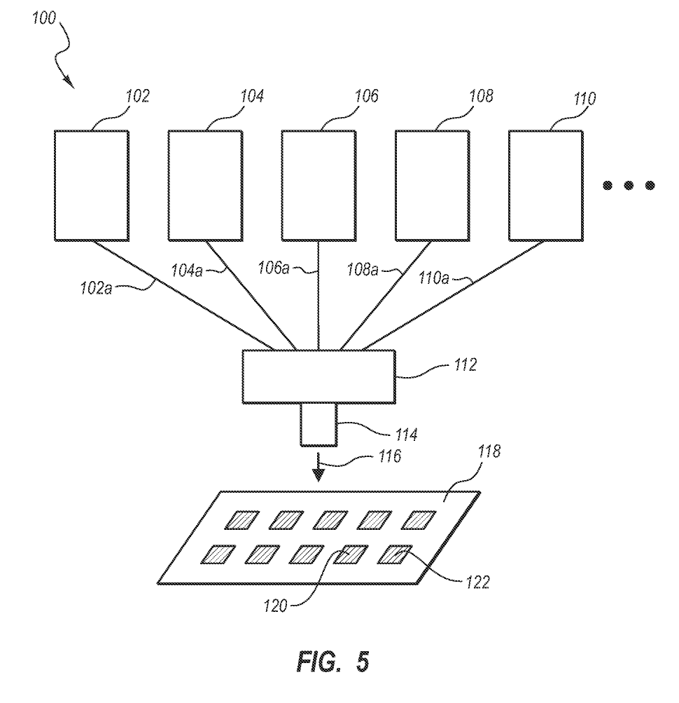

FIG. 5 is a schematic illustration of an inkjet printing system 100 configured to print a composition onto a flexible substrate 118. Such an inkjet printing system can include any one of or combination of the following: an inkjettable sensor solution 102 having a sensor, such as a nanosensor; an inkjettable nanowire solution 104 having a nanowire; an inkjettable pre-nanowire solution 106 having pre-nanowire components that can be printed into a nanowire; an inkjettable conducting polymer solution 108 having pre-conducting polymer components that can be printed into a conducting polymer; an inkjettable pre-conducting polymer solution 110 having monomers or polymers of a conducting polymer that can be inkjet printed; and any other suitable inkjetting solution. Additional solutions can include: inkjet ink for printing indicia on the flexible substrate; an inkjettable binder solution to bind the sensor or sensor circuit to the flexible substrate, where the binder can be similarly flexible; an inkjettable metallic composition including metallic particulates that can be printed into electronic pathways; or any other inkjettable composition. The inkjet printing system 100 is also shown to include: a fluid conduit 102a for the inkjettable sensor solution 102; a fluid conduit 104a for the inkjettable nanowire solution 104; a fluid conduit 106a for the inkjettable pre-nanowire solution 106; a fluid conduit 108a for the inkjettable conducting polymer solution 108; and a fluid conduit 110a for the inkjettable pre-conducting polymer solution 110. The fluid conduits can couple the inkjettable solutions to an inkjet printer 112 and to a printer head 114 that can inkjet print 116 one of the compositions onto a flexible substrate 118. The inkjet printer 112 then prints 116 the compositions into a sensor 120, sensor circuit 122 (FIG. 2), or combination thereof.

In one embodiment, a method of manufacturing a flexible sensor device can include inkjetting a nanosensor-containing composition onto a flexible substrate so as to deposit and retain one or more of nanosensors in a first predetermined pattern of a first macrosensor on the flexible substrate. The flexible substrate that has inkjet-printed nanosensors can be configured to have a desired degree of elongation, contraction and distortion while retaining sensing functions of the nanosensors. Such configuration can be achieved by the flexible substrate having such flexibility. Also, the inkjetted composition can include components, such as binders, elastomers, polymers, or the like, that provide post printing flexibility.

The sensor or macrosensor formed from inkjetting can include a number of nanosensors or sensors. For example, the first predetermined pattern of the sensor or macrosensor can include about or at least about 10 nanosensors or sensors, at least about 50 nanosensors or sensors, at least about 100 nanosensors or sensors, at least about 1,000 nanosensors or sensors, at least about 10,000 nanosensors or sensors, at least about 100,000 nanosensors or sensors, at least about 1 million nanosensors or sensors, at least about 10 million nanosensors or sensors, least 100 million nanosensors or sensors, or at least 1 billion nanosensors or sensors. Also, the sensor device can include from about 1 nanosensor about 10 nanosensors, from about 10 nanosensors to about 50 nanosensors, from about 50 nanosensors to about 100 nanosensors, from about 100 nanosensors to about 1,000 nanosensors, from about 1,000 nanosensors to about 10,000 nanosensors, from about 10,000 nanosensors to about 100,000 nanosensors, from about 100,000 nanosensors to about 1 million nanosensors, from about 1 million nanosensors to about 10 million nanosensors, from about 10 million nanosensors to about 100 million nanosensors, or from 100 million nanosensors to about 1 billion nanosensors. As described, a printed sensor or macrosensor can include an individual sensor or nanosensor or the large numbers of sensors or nanosensors. The difference between sensors and nanosensor can be based on size or the like.

In one embodiment, the method of manufacture can include inkjetting a second nanosensor-containing composition onto the flexible substrate. The second nanosensor-containing composition can include nanosensors that are different from the other nanosensors. The nanosensors can be configured to detect different target substances. Alternatively, the nanosensors can be a different type that detect the same target substance.

In one embodiment, manufacturing can include inkjetting a conducting polymer-containing composition onto the flexible substrate so as to form a sensor circuit that is operably coupled with at least one inkjet printed nanosensor. The sensor circuit can include circuit components formed from the conducting polymer. The inkjetting of the conducting polymer-containing composition can also include the inkjetting of components that form a conducting polymer, such as, monomers, polymerizers, dopants, reactants, binders, polymers, conductive components, metallic components, and the like that can form a conducting polymer in a circuit configuration. Thus, the printing of a conducting polymer can be performed by printing components that combine to form a conducting polymer on the substrate.

In one embodiment, manufacturing can include inkjetting a nanowire complex-containing composition onto the flexible substrate so as to form a sensor circuit that is operably coupled with at least one inkjet printed nanosensor. The sensor circuit can include circuit components formed from the nanowire. The inkjetting of the nanowire-containing composition can also include the inkjetting of components that form a nanowire, such as, semiconductor materials, monomers, polymerizers, dopants, reactants, binders, polymers, and the like that can form a nanowire in a circuit configuration. Thus, the printing of a nanowire polymer can be performed by printing components that combine to form a conducting polymer on the substrate.

In one embodiment, manufacturing can include inkjetting a conducting polymer-containing composition and a nanowire complex-containing composition onto the flexible substrate so as to form a sensor circuit that is operably coupled with at least one inkjetted nanosensor. The conducting polymer and nanowire complex can cooperate to form the sensor circuit. The conducting polymer-containing composition can be retained in a separate reservoir from the nanowire complex-containing composition. As before, the formation of the sensor circuit can be performed by printing pre-conducting polymer components and/or pre-nanowire components that form conducting polymers and/or nanowires on the substrate so as to form the sensor circuit.

In one embodiment, the flexible substrate can be incorporated into a wearable garment. Wearable garments that include sensors can be used for sensing biometric data as well as sensing target substances as described herein. In some instances, the biometric data can be obtained from detecting target substances. As such, the method of manufacture can include configuring the flexible substrate having the inkjet-printed nanosensors with sufficient flexibility for being a component of a wearable garment such that the macrosensor is capable of sensing biometric data of a subject wearing the wearable garment. The sensors can detect a chemical that is provided from a subject wearing the garment, and the detection of the chemical or determination of the amount or concentration of the chemical in or on the subject can provide biometric data. Biometric data can then be used for health purposes and/or determine the health state of the subject.

In one embodiment, a nanosensor-containing composition can be inkjetted onto the flexible substrate so as to deposit and retain one or more of nanosensors in at least a second predetermined pattern of at least a second macrosensor on the flexible substrate. The first and second macrosensors can be separated by cutting the flexible substrate. Alternatively, the first macrosensor can be placed onto the second macrosensor and the flexible substrate can be adhered together to form a pouch having both macrosensors. Also, this can include operably coupling a second macrosensor with the first macrosensor.

The method of manufacture can include placing a second flexible substrate onto the flexible substrate having the inkjet-printed nanosensors, and bonding the second flexible substrate to the flexible substrate having the inkjet-printed nanosensors. This can be used to prepare the sensor devices as described herein. Also, the flexible substrate can be folded onto itself and bonded to form a container as described herein.

Accordingly, a method of preparing a flexible sensor device by inkjet printing can include inkjetting a sensor-containing composition onto a flexible substrate so as to deposit and retain one or more sensors in a first predetermined pattern of a first sensor (e.g., macrosensor) on the flexible substrate. The inkjet printed sensor can have the flexibility, elongation, contraction, and/or distortion properties as described herein. The flexible substrate having the inkjet-printed sensors is configured to have a desired degree of elongation, contraction, and distortion while retaining sensing functions of the sensors. Also, the inkjetted composition can include components, such as binders, elastomers, polymers, or the like, that provide post printing flexibility.

In one embodiment, the method of manufacturing a flexible sensor device can also include any one or combination of the following: inkjetting a second sensor-containing composition onto the flexible substrate; inkjetting a conducting polymer-containing composition onto the flexible substrate so as to form a sensor circuit that is operably coupled with at least one inkjet printed sensor; inkjetting a nanowire complex-containing composition onto the flexible substrate so as to form a sensor circuit that is operably coupled with at least one inkjet printed sensor; inkjetting a nanowire complex-containing composition onto the flexible substrate so as to form a sensor circuit that is operably coupled with at least one inkjetted sensor and the inkjetted nanowire complex containing sensor circuit, wherein the conducting polymer-containing composition is retained from a separate reservoir from the nanowire complex-containing composition; or inkjetting a sensor-containing composition onto the flexible substrate so as to deposit and retain a plurality of sensors in at least a second predetermined pattern of at least a second macrosensor on the flexible substrate; or operably coupling a second macrosensor with the first sensor (e.g., first macrosensor). Such manufacturing steps can be performed as described herein or known in the art. The printed sensors can be individual sensor or any number of sensors together so as to form a macrosensor. Macrosensors are considered to be a sensor formed of sensors and/or nanosensors.

In one embodiment, a method of manufacturing a flexible sensor device having one or more sensor circuits by inkjet printing. The inkjet printing method can include inkjetting at least one composition having components for forming a sensor circuit onto a flexible substrate so as to form and retain at least one sensor circuit on the flexible substrate in a predetermined pattern. The sensor circuit can be configured for sensing an interaction with a target substance. The flexible substrate having the inkjet-printed sensor circuit can be configured to have a desired degree of elongation, contraction, and distortion while retaining sensing functions of the sensor circuit.

In one embodiment, the method of manufacture can also include any of the following: preparing the at least one composition having components for forming the sensor circuit to have a conducting polymer-containing composition configured for being inkjetted onto the flexible substrate; preparing the at least one composition having components for forming the sensor circuit to include a nanowire complex-containing composition configured for being inkjetted onto the flexible substrate; inkjetting a conducting polymer-containing composition onto the flexible substrate so as to form the sensor circuit; inkjetting a nanowire complex-containing composition onto the flexible substrate so as to form the sensor circuit; inkjetting a conducting polymer-containing composition and a nanowire complex-containing composition onto the flexible substrate so as to form the sensor circuit; inkjetting a nanosensor-containing composition onto the flexible substrate so as to deposit and retain a plurality of nanosensors in a first predetermined pattern of a first macrosensor on the flexible substrate, said flexible substrate having the inkjet-printed nanosensors being configured to have a desired degree of elongation, contraction and distortion while retaining sensing functions of the nanosensors, the first macrosensor being operably coupled with the at least one sensing circuit and being configured to interact with a target substance; or configuring the flexible substrate having the inkjet-printed nanosensors with sufficient flexibility for being a component of a wearable garment such that the macrosensor is capable of sensing biometric data of a subject wearing the wearable garment. Also, the method can include placing a second flexible substrate onto the flexible substrate having the inkjet-printed sensor circuit, and bonding the second flexible substrate to the flexible substrate having the inkjet-printed sensor circuit. Such manufacturing steps can be performed as described herein or known in the art.

In one embodiment, a system for manufacturing a flexible sensor device. Such a system can include any combination of the printing system, inkjet printer, compositions, and/or other features described herein for inkjet printing onto a flexible substrate in order to prepare a flexible sensor device.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein in their entirety by specific reference.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A flexible sensor device comprising:
a flexible substrate; and
at least one flexible sensor included on the flexible substrate.

2. A sensor device as in claim 1, wherein the flexible sensor is configured to function when subjected to elongation, contraction, and/or distortion.

3. A sensor device as in claim 1, wherein the flexible sensor is a macrosensor that includes one or more sensors.

4. A sensor device as in claim 1, wherein the flexible sensor includes one or more nanosensors.

5. A sensor device as in claim 1, wherein the flexible sensor is configured to detect a target substance so as to provide a detectable signal.

6. A sensor device as in claim 5, wherein the target substance include an organic molecule, inorganic molecule, atom, ion, nucleotide, polynucleotide, amino acid, polypeptide, protein, receptor, antibody, antibody fragment, cell, cell surface component, ligand, or combinations thereof.

7. A sensor device as in claim 1, wherein the flexible sensor is a flexible sensor circuit.

8. A sensor device as in claim 7, wherein the flexible sensor circuit includes at least one of a nanowire or a conducting polymer.

9. A sensor device as in claim 8, wherein the flexible sensor circuit includes at least one nanowire and at least one conducting polymer.

10. A sensor device as in claim 1, wherein the flexible substrate having the inkjetted sensor is configured with sufficient flexibility for being a component of a wearable garment such that the sensor is capable of sensing a target substance that provides biometric data of a subject wearing the wearable garment.

11. A method of manufacturing a flexible sensor device, the method comprising:
selecting a flexible substrate; and
inkjetting at least one flexible sensor onto the flexible substrate.

12. A method as in claim 11, further comprising configuring the flexible substrate having the flexible sensor to function as a sensor device when subjected to elongation, contraction, and/or distortion.

13. A method as in claim 11, further comprising inkjetting a plurality of sensors onto the flexible substrate to form a flexible macrosensor.

14. A method as in claim 1, further comprising inkjetting one ore more nanosensors onto the flexible substrate.

15. A method as in claim 1, further comprising configuring the flexible sensor to detect a target substance so as to provide a detectable signal.

16. A method as in claim 15, further comprising selecting a target substance to detect, said target substance including an organic molecule, inorganic molecule, atom, ion, nucleotide, polynucleotide, amino acid, polypeptide, protein, receptor, antibody, antibody fragment, cell, cell surface component, ligand, or combinations thereof.

17. A method as in claim 11, further comprising forming the flexible sensor into a flexible sensor circuit.

18. A method as in claim 17, further comprising inkjetting one of a nanowire or a conducting polymer onto the flexible substrate to form the flexible sensor circuit.

19. A method as in claim 17, further comprising inkjetting a nanowire and a conducting polymer onto the flexible substrate to form the flexible sensor circuit.

20. A method as in claim 11, further comprising configuring the flexible substrate having the flexible sensor with sufficient flexibility for being a component of a wearable garment such that the sensor is capable of sensing a target substance that provides biometric data of a subject wearing the wearable garment.
